(19) United States
(12) Patent Application Publication
(10) Pub. No.: US 2005/0287212 A1
(43) Pub. Date: Dec. 29, 2005

(54) Oral delivery system comprising a drug/polymer complex

(76) Inventors: Liang-Chang Dong, Sunnyvale, CA (US); Jasmine E. Han, San Jose, CA (US); Crystal Pollock-Dove, Mountain View, CA (US); Patrick S.L. Wong, Burlingame, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/148,673

(22) Filed: Jun. 8, 2005

Related U.S. Application Data
(60) Provisional application No. 60/583,816, filed on Jun. 28, 2004.

(51) Int. Cl. A61K 9/48; A61K 9/24

(52) U.S. Cl. 424/473

(57) ABSTRACT

This invention pertains to the enhanced delivery of orally administered pharmaceutical agents and methods, dosage forms and devices thereof. In particular, the invention is directed to methods including providing a low solubility drug having a pKa between about 6 and about 9; dissolving the low solubility drug in an aqueous solution, wherein a pH of the aqueous solution is less than about 6.0; dissolving a hydrophilic polymer in the aqueous solution, wherein the weight ratio of the hydrophilic polymer to the low solubility drug is less than or equal to about 0.15; lyophilizing the aqueous solution to obtain a lyophilized powder. Also disclosed are drug formulations made according to the method, and dosage forms that include the drug formulations.

### CROSS-REFERENCE TO RELATED APPLICATION

This application claims benefit, under 35 U.S.C 119(e), of U.S. Ser. No. 60/583,816, filed Jun. 28, 2004, which is incorporated herein by reference.

### FIELD OF THE INVENTION

This invention pertains to the enhanced delivery of orally administered pharmaceutical agents and methods, dosage forms and devices thereof. In particular, the invention is directed to drug formulations, dosage forms and methods for enhancing controlled delivery of low solubility drugs having a pKa between about 6 and about 9.

### BACKGROUND OF THE INVENTION

The art is replete with descriptions of dosage forms for release of pharmaceutical agents. A variety of dosage forms for delivering certain drugs may be known, but not every drug may be suitably delivered from those dosage forms. For example, solubility parameters may be unique to a particular drug and/or its mode of delivery. This is a particular concern with drugs that are more soluble in low pH gastric fluid than in neutral pH intestinal fluid.

For this reason, dosage forms that incorporate such low solubility drugs provide a major challenge for sustained release technologies.

In situations requiring multiple doses of a particular drug, the blood level of a drug often needs to be maintained above a minimum effective level and below a minimum toxic level to achieve optimal efficacy and safety. A controlled drug delivery system is usually designed to deliver the drug at this particular rate; a safe and effective blood level is maintained for as long as the system continues to deliver the drug at this rate. A controlled release dosage form ideally should provide a relatively constant blood level of active ingredient, and avoid the sharp fluctuations observed when multiple doses of immediate release dosage forms are administered. By achieving a constant blood level, a drug's benefit is maximized while its potential toxic effects are minimized.

An orally administered controlled drug delivery system encounters a wide range of highly variable conditions moving from the highly acidic environment of the stomach to the neutral and basic conditions of the intestinal lumen. Ideally, an oral controlled drug delivery system will not only deliver a drug at a constant and reproducible rate within this range of conditions, but the drug will remain in solution until absorption occurs.

A variety of dosage forms provide constant release of a drug over several hours. While dosage forms can deliver a drug composition to the environment of use, after release from the dosage form, a low solubility drug may precipitate during its transit through the environment of neutral pH. Accordingly, it may be advantageous to release the low solubility drug in the form that will remain in solution. This is particularly important for Class II drugs that have low solubility in aqueous solution and a high rate of absorption by the GI tract.

It is a great challenge to develop oral dosage forms for certain drugs that are soluble at the low pH environment of gastric fluid precipitate out of solution upon entry to the small intestine where the pH is neutral. This precipitation causes erratic absorption of these drugs. There is an unmet need for a new oral dosage form for this class of drugs.

An example of such a drug is ciprofloxacin. This drug has been developed into a commercial product, CIPRO® XR, and is described in U.S. Pat. No. 6,261,601 and published application WO 01/64183. While the commercial ciprofloxacin products are designed to delivery high doses of drug, the problem of drug precipitation at neutral pH remains unsolved. Further, the commercial controlled release dosage form produces acute fluctuations of drug levels characteristic of immediate release formulations. Moreover, these fluctuations are greater in amplitude than those associated with immediate release dosage forms. A demand remains for a dosage form that will provide a drug at a sustained, constant level in solution in the neutral and basic pH conditions of the intestinal lumen over the full dosage period.

### SUMMARY OF THE INVENTION

In an aspect, the invention relates to a method comprising: providing a low solubility drug having a pKa between about 6 and about 9; dissolving the low solubility drug in an aqueous solution, wherein a pH of the aqueous solution is less than about 6.0; dissolving a hydrophilic polymer in the aqueous solution; and lyophilizing the aqueous solution to obtain a lyophilized powder, wherein the weight ratio of the hydrophilic polymer to the low solubility drug in the lyophilized powder is less than or equal to about 0.15.

### BRIEF DESCRIPTION OF THE DRAWINGS

FIG. 1 shows the structure of the complex between a drug and a hydrophilic polymer according to the invention. The basic drug forms a monomeric complex with the hydrophilic polymer. This monomeric drug complex may aggregate, forming a micelle-like structure (lower left) which can absorb and solubilize more drug (lower right).

FIG. 2 shows the results of precipitation studies of ciprofloxacin with various hydrophilic polymers. Ciprofloxacin and various hydrophilic polymers dissolved in deionized water were added to artificial intestinal fluid (AIF). The precipitation study was conducted using the Distek USP II method. The amount of drug dissolved was measured by absorbance at 323 nm.

FIG. 3 shows the results of precipitation studies of ciprofloxacin formulations comprising different excipients. The composition of each formulation is listed in Table 2. Error bars represent the standard deviation of 2 or 3 experiments.

FIG. 4 shows the results of precipitation studies of ciprofloxacin with different excipients in dry-blended (DB) formulations. Error bars represent the standard deviation of 2 experiments. The DB ciprofloxacin mixtures were added to 900 ml AIF in 100 rpm.

FIG. 5 shows the results of precipitation studies of ciprofloxacin/HPMC in freeze-dried (i.e. lyophilized) (FD) mixtures containing various excipients. Error bars represent the standard deviation of 2 or 3 experiments. The FD ciprofloxacin mixtures were added to 900 ml AIF in 100 rpm, and absorbance was monitored.

FIG. 6 shows the results of precipitation studies of FD and DB ciprofloxacin/HPMC mixtures. Error bars represent the standard deviation of 2 or 3 experiments. The ciprofloxacin mixtures were added to 900 ml AIF in 100 rpm, and absorbance was monitored.

FIG. 7 shows the FTIR spectra of ciprofloxacin/HPMC, 50:50, wt:wt, in FD and DB formulations.

FIG. 8 shows the chemical structure of ciprofloxacin.

FIG. 9 shows the chemical structure of HPMC.

FIG. 10 shows the FTIR spectra of ciprofloxacin alone, HPMC alone and ciprofloxacin/HPMC in a 50:50 weight ratio, in a DB formulation.

FIG. 11 shows the FTIR spectra of ciprofloxacin:HPMC in various weight ratios: FD, 90:10 and 50:50; and DB, 50:50.

FIG. 12 shows the Raman spectra of ciprofloxacin/HPMC mixtures: ciprofloxacin alone; HPMC alone; and various weight ratios, ciprofloxacin:HPMC: FD, 90:10 and 50:50; and DB, 90:10 and 50:50.

FIG. 13 shows the results of precipitation studies of ciprofloxacin/HPMC tablet formulations with and without circulation pumping solutions within vessels.

FIG. 14 shows the results of precipitation studies of ciprofloxacin/HPMC (90:10) tablet formulations under different circulation pumping conditions: less circulation and no pump back; less circulation and pump back and more circulation and pump back.

### DETAILED DESCRIPTION OF THE INVENTION

The present invention goes to solving the unmet problem of providing constant levels of drug in the intestinal fluid over an extended period of time. The present invention provides a means to control of the delivery pattern of drugs having low solubility in the intestinal fluid.

### DEFINITIONS

All documents cited to herein are incorporated by reference as if reproduced fully herein.

By “dosage form” is meant a pharmaceutical composition or device comprising an active pharmaceutical agent, such as ciprofloxacin or a pharmaceutically-acceptable acid addition salt thereof, a structural polymer, a solubilizing surfactant and the composition or device optionally containing inactive ingredients, i.e., pharmaceutically acceptable excipients such as disintegrants, binders, diluents, lubricants, stabilizers, antioxidants, osmotic agents, colorants, plasticizers, coatings and the like, that are used to manufacture and deliver active pharmaceutical agents.

By “active agent”, “drug”, or “therapeutic agent” is meant an agent, drug, or compound having therapeutic characteristics or a pharmaceutically-acceptable acid addition salt thereof.

By “pharmaceutically-acceptable acid addition salt” or “pharmaceutically acceptable salt”, which are used interchangeably herein, are meant those salts in which the anion does not contribute significantly to the toxicity or pharmacological activity of the salt, and, as such, they are the pharmacological equivalents of the bases of the compound. Examples of pharmaceutically acceptable acids that are useful for the purposes of salt formation include but are not limited to hydrochloric, hydrobromic, hydroiodic, citric, succinic, tartaric, maleic, acetic, benzoic, mandelic, phosphoric, nitric, mucic, isethionic, palmitic, and others.

By “low solubility” is meant that the neat therapeutic agent exhibits solubility in AIF (pH 6.8) that is less than in AGF (pH 1.2) or other acedic media. Aqueous solubility is determined by adding the therapeutic agent to stirred or agitated medium maintained in a constant temperature bath at a temperature of 37 degrees centigrade until equilibrium is established between the dissolved and undissolved states and the concentration of dissolved drug is constant. The resulting solution saturated with active agent is then filtered, typically under pressure through a 0.8-micron Millipore filter, and the concentration in solution is measured by any appropriate analytical method including gravimetric, ultraviolet spectrophometry, chromatography.

By “sustained release” is meant predetermined continuous release of active agent to an environment over a prolonged period.

The expressions “drug delivery orifice,” and other similar expressions, as may be used herein include a member selected from the group consisting of a passageway; an aperture; an orifice; and a bore. The expression also includes an orifice that is formed or formable from a substance or polymer that erodes, dissolves or is leached from the outer wall to thereby form an exit orifice.

An “immediate-release dosage form” refers to a dosage form that releases drug substantially completely within a short time period following administration, i.e., generally within a few minutes to about 1 hour.

By “controlled release dosage form” is meant a dosage form that releases drug substantially continuously for many hours. Controlled release dosage forms in accord with the present invention release drug for at least about 8 to 20 hours and preferably 15 to 18 hours and more preferably about 17 hours or more. The dosage forms continuously release drug for sustained periods of at least about 8 hours, preferably 12 hours or more and, more preferably, 16-20 hours or more.

Dosage forms in accord with the present invention exhibit controlled release rates of a therapeutic agent for a prolonged period of time within the sustained release time period.

By “uniform release rate” is meant an average hourly release rate from the core that varies positively or negatively by no more than about 30% and preferably no more than about 25% and most preferably no more than 10% from either the preceding or the subsequent average hourly release rate as determined in a USP Type VII Interval Release Apparatus where the cumulative release is between about 25% to about 75%.

By “prolonged period of time” is meant a continuous period of time of at least about 4 hours, preferably 6-8 hours or more and, more preferably, 10 hours or more. For example, the exemplary osmotic dosage forms described herein generally begin releasing therapeutic agent at a uniform release rate within about 2 to about 6 hours following administration and the uniform rate of release, as defined above, continues for a prolonged period of time from about 25% to until at least about 75% and preferably at least about 85% of the drug is released from the dosage form. Release of therapeutic agent continues thereafter for several more hours although the rate of release is generally slowed somewhat from the uniform release rate.

Low Solubility Drug Formulation

In this invention, a low solubility drug and a hydrophilic polymer are formed into a lyophilized powder. Preferably, the low solubility drug possesses a pKa between about 6 and about 9. Upon solubilization in aqueous solutions at neutral pH, such as AIF, the lyophilized powder may form micelles in which hydrophobic areas of the drug molecules aggregate and the hydrophilic regions complex with the hydrophilic polymers. Such aqueous solutions may prevent or reduce precipitation of these low solubility drugs at neutral pH. In an embodiment, the measured precipitation of the low solubility drug from such aqueous solutions is less than precipitation of the low solubility drug obtained when the low solubility drug is dissolved directly in a second aqueous solution having a pH approximately equal to, or equal to, the first aqueous solution. The low solubility drug is not added to the second aqueous solution in the form of the lyophilized powder, rather it is generally added alone (i.e. the drug substances is simply added to the aqueous solution in a conventional manner).

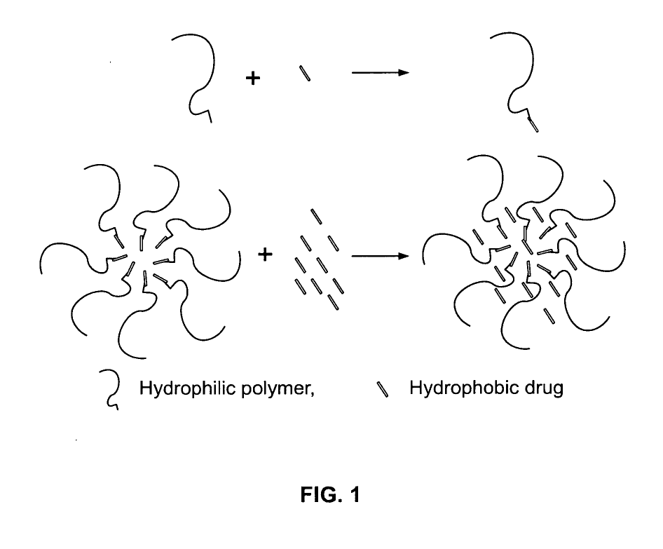

FIG. 1 depicts the structure of the complex, which is based on hydrogen bonding between drug molecules and hydrophilic polymer molecules. The basic drug hydrogen bonds with the hydrophilic polymer to form a monomeric complex. This monomeric complex aggregates forming a micelle-like structure having a hydrophilic exterior in contact with water, and a hydrophobic interior. The hydrophobic interior can absorb and solubilize more drug moleules.

The present invention provides a drug formulation for enhanced uptake of a therapeutic drug, comprising a low solubility drug and a hydrophilic polymer, in which the drug is more soluble in low pH gastric fluid than in neutral pH intestinal fluid, and in which the polymer complexes with the drug in artificial intestinal fluid (AIF) and inhibits its precipitation. Precipitation, and inhibition or reduction thereof, is preferably measured using the Distek USP II method. In another embodiment, the invention provides a method comprising providing a low solubility drug having a pKa between about 6 and about 9; dissolving the low solubility drug in an aqueous solution, wherein a pH of the aqueous solution is less than about 6.0; dissolving a hydrophilic polymer in the aqueous solution, wherein the weight ratio of the hydrophilic polymer to the low solubility drug is less than or equal to about 0.15; and lyophilizing the aqueous solution to obtain a lyophilized powder. In a preferred embodiment, the invention further provides the above method which further comprises dissolving the lyophilized powder in a first aqueous solution at pH greater than about 6.0; measuring precipitation of the low solubility drug from the first aqueous lyophilized powder solution; wherein the measured precipitation is less than precipitation of the low solubility drug obtained when the low solubility drug is dissolved directly in a second aqueous solution having a pH equal to the first aqueous solution. Preferably the first and second aqueous solution comprise the same or similar ingredients except for the lyophilized powder and/or the low solubility drug. Dissolving the low solubility drug directly in an aqueous solution means adding the low solubility drug to the aqueous solution, but not in the form of the low solubility drug-containing lyophilized powder.

This invention applies to a wide range of low solubility drugs. In embodiments, these are basic drugs, i.e., ones having a pKa greater than 6.5. Preferably, these are compounds having a pKa between 6 and 9, most preferably between 6.5 and 7.5. Most specifically, the low solubility drug is one selected from the group consisting of ciprofloxacin, phenytoin, acyclovir, alprenolol, atenolol, azithromycin, buspirone, carvedilol, diltiazem, imipramine, metoprolol, normorphine, oleandomycin, paromomycin, theophyline and vancomycin.

In an embodiment, the class of drugs to which this invention applies can be operationally defined with respect to its tendency to precipitate at neutral pH and above in a dissolution (precipitation) test. Preferably, the class of drugs that precipitate in aqueous solvents between pH 6 and 9, most preferably between 6.5 and 7.5.

Accordingly, in an embodiment, the drugs to which this invention applies are ones that would be predicted to precipitate within the lumen of the intestine. Precipitation of these drugs in the lumen causes these drugs to have low uptake in orally administered formulations.

According to the present invention, the recited hydrophilic polymers may form a complex with the low solubility drug. The presence of the complexes can be shown in numerous ways. Several methods are presented herein.

In one methodology, the complex can be measured by FTIR spectroscopy. In an embodiment, the FTIR absorbance bands of the low solubility drug present in the lyophilized powder are shifted compared to the low solubility drug alone. By “the low solubility drug alone” is meant the low solubility drug in its neat, or bulk form, and not in the form of the lyophilized powder. For example, formation of an inventive lyophilized powder with a hydrophilic polymer causes the shift in ciprofloxacin's absorbance in the 1705, 2507 or 2470 cm⁻¹ absorbance bands by FTIR.

In another methodology, the complex can measured by Raman spectroscopy. In an embodiment, the Raman absorbance bands of the low solubility drug present in the lyophilized powder are shifted compared to the low solubility drug alone. By “the low solubility drug alone” is meant the low solubility drug in its neat, or bulk form, and not in the form of the lyophilized powder. For example, formation of an inventive lyophilized powder with a hydrophilic polymer causes a shift in the 1709, 1549, 1388 or 1347 cm⁻¹ absorbance bands of ciprofloxacin by Raman spectroscopy.

In both methodologies, formation of complexes can be measured by comparing the spectra of the drug in the presence and absence of the hydrophilic polymer. If a difference is observed, the peaks can be assigned to particular chemical groups, including amines, carbonyls, alcohols and esters. The relevance of a shift can be evaluated with respect to a tendency for the hydrophilic polymer to hydrogen bond with the drug.

The tendency for the complex to be disrupted by shear force is another measure of its presence in a solution. For example, one can measure the ability of a drug to precipitate in AIF before and after applying a shear force in the dissolution tank. If shear forces accelerate precipitation of the drug, this is confirmatory evidence for the presence of the micelle-forming complex.

The hydrophilic polymer alternatively, can be selected from the group consisting of hydroxypropyl methylcellulose, polyvinyl alcohol-polyethylene glycol graft copolymer and ethylene oxide/propylene oxide copolymer, methylcellulose (including Methocel OS cellulose ether, Methocel SG A 150, Methocel SG A7C AND Methocel SG A16M).

The drug formulation may be prepared by a process comprising mixing the low solubility drug with a hydrophilic polymer in an aqueous solution, dissolving the drug and the polymer in the solution, and lyophilizing the solution to obtain a dried powder.

Alternatively, the drug formulation may be prepared by a process comprising mixing the low solubility drug with the hydrophilic polymer and adipic acid, and dry-blending the mixture.

An advantage of the present invention is that higher low solubility drug loadings are possible in dosage forms as compared to alternative formulation strategies such as conventional solid dispersions. In an embodiment, the weight ratio of the hydrophilic polymer to the low solubility drug in the lyophilized powder is less than or equal to about 0.15, more preferably the weight ratio of the hydrophilic polymer to the low solubility drug in the lyophilized powder is less than or equal to about 0.10, even more preferably the weight ratio of the hydrophilic polymer to the low solubility drug in the lyophilized powder is less than or equal to about 0.08.

Low Solubility Drug Dosage Form

The lyophilized powder, and/or pharmaceutical formulations comprising the lyophilized powder, can be incorporated into a matrix or OROS osmotic system (or other controlled release dosage forms known in the art) to achieve a controlled release dosage form.

The push layer comprises a displacement composition in a layered arrangement with the drug layer. The push layer comprises an osmopolymer that imbibes an aqueous or biological fluid and swells to push the drug composition through the exit means of the device. A polymer having suitable imbibition properties may be referred to herein as an osmopolymer. The osmopolymers are swellable, hydrophilic polymers that interact with water and aqueous biological fluids and swell or expand to a high degree, typically exhibiting a 2-50 fold volume increase. The osmopolymer can be non-crosslinked or crosslinked.

The push layer comprises 20 to 375 mg of the osmopolymer. Representatives of fluid-imbibing displacement polymers comprise members selected from poly(alkylene oxide) of 1 million to 15 million number-average molecular weight, as represented by poly(ethylene oxide), and poly(alkali carboxymethylcellulose) of 500,000 to 3,500,000 number-average molecular weight, in which the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

The push layer comprises 0 to 75 mg, and presently 5 to 75 mg of an osmotically effective compound, an osmoagent. The osmotically effective compounds are known also as osmoagents and as osmotically effective solutes. Osmoagent that may be found in the drug layer and the push layer in the dosage form are those that exhibit an osmotic activity gradient across a wall of the dosage form. Suitable osmoagents comprise a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts and carbohydrates.

The push layer may further comprise a therapeutically acceptable vinyl polymer. The vinyl polymer comprises a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Push layer contains 0.01 to 25 mg of vinyl polymer.

The push layer may further comprise 0 to 5 mg of a nontoxic colorant or dye. Colorants includes Food and Drug Administration Colorant (FD&C), such as FD&C No. 1 blue dye, FD&C No. 4 red dye, red ferric oxide, yellow ferric oxide, titanium dioxide, carbon black, and indigo.

The push layer may further comprise a lubricant. Typical lubricants comprise a member selected from the group consisting of sodium stearate, potassium stearate, magnesium stearate, stearic acid, calcium stearate, sodium oleate, calcium palmitate, sodium laurate, sodium ricinoleate and potassium linoleate, and blends of such lubricants. The amount of lubricant included in the push layer is 0.01 to 10 mg.

The push layer may further comprise an antioxidant to inhibit the oxidation of ingredients comprising expandable formulation. The push layer comprises 0.00 to 5 mg of an antioxidant. Representative antioxidants comprise a member selected from the group consisting of ascorbic acid, ascorbyl palmitate, butylated hydroxyanisole, a mixture of 2 and 3 tertiary-butyl-4-hydrowynisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

The present invention may comprise an overcoat of drug on the dosage form. An overcoat is a therapeutic composition comprising a mixture of drug and a pharmaceutically acceptable carrier selected from the group consisting of alkylcellulose, hydroxyalkylcellulose and hydroxypropylalkylcellulose. The overcoat is represented by methylcellulose, hydroxyethylcellulose, hydroxybutylcellulose, hydroxypropylcellulose, hydroxypropylmethylcellulose, hydroxypropylethylcellulose and hydroxypropylbutylcellulose, polyvinyl pyrrolidone/vinyl acetate copolymer, polyvinyl alcohol-polyethylene graft copolymer, and the like. The overcoat provides therapy immediately as it dissolves or undergoes dissolution in the presence of gastrointestinal fluid and concurrently delivers drug into the gastrointestinal tract for immediate therapy.

The semipermeable wall is formed to be permeable to the passage of an external fluid, such as water and biological fluids, and it is substantially impermeable to the passage of drug, osmagent, osmopolymer and the like. As such, it is semipermeable. The selectively semipermeable compositions used for forming the wall are essentially nonerodible and they are substantially insoluble in biological fluids during the life of the dosage form.

Representative polymers for forming the wall comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, in which the organic moieties contain from one to twelve carbon atoms, and preferably from one to eight carbon atoms.

The semipermeable compositions typically include a member selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Additional semipermeable polymers for forming the outer wall comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyitrimethylammonium chloride); and semipermeable polymers exhibiting a fluid permeability of 10⁻⁵ to 10⁻² (cc. mil/cm hr.atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio. Wall 20 can optionally be formed as two or more lamina such as described in U.S. Pat. No. 6,210,712.

The wall may also comprise a flux-regulating agent. The flux regulating agent is a compound added to assist in regulating the fluid permeability or flux through the wall. The flux-regulating agent can be a flux-enhancing agent or a flux-decreasing agent. The agent can be preselected to increase or decrease the liquid flux. Agents that produce a marked increase in permeability to fluid such as water are often essentially hydrophilic, while those that produce a marked decrease to fluids such as water are essentially hydrophobic. The amount of regulator in the wall when incorporated therein generally is from about 0.01% to 30% by weight or more. The flux regulator agents may include polyhydric alcohols, polyalkylene glycols, polyalkylenediols, polyesters of alkylene glycols, and the like. Typical flux enhancers include polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Presently preferred flux enhancers include the group of difunctional block-copolymer polyoxyalkylene derivatives of propylene glycol known as Lutrols. Representative flux-decreasing agents include phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; polyvinyl acetates, triethyl citrate, Eudragit; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials may be included in the semipermeable wall material for imparting flexibility and elongation properties, to make the wall less brittle and to render tear strength. Suitable materials include phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

Pan coating may be conveniently used to provide the walls of the completed dosage form. In the pan coating system, the wall-forming composition for the wall is deposited by successive spraying of the appropriate wall composition onto the compressed single or bilayered core comprising the drug layer for the single layer core or the drug layer and the push layer for the laminated core, accompanied by tumbling in a rotating pan. A pan coater is used because of its availability at commercial scale. Other techniques can be used for coating the compressed core. Once coated, the wall is dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the wall or walls of the dosage form may be formed in one technique using the air-suspension procedure. This procedure consists of suspending and tumbling the compressed single or bilayer core in a current of warmed air and the semipermeable wall forming composition, until the wall is applied to the core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent for the wall forming material. An Aeromatic® air-suspension coater can be used employing a cosolvent.

The drug is exemplified herein through the use of ciprofloxacin, which is more soluble in low pH gastric fluid than in neutral pH intestinal fluid and therapeutically required to be delivered in high doses, e.g., 200 to 1500 mg.

The solubility of ciprofloxacin is pH sensitive: in AGF (pH 1.2), the solubility of ciprofloxacin is approximately 30 mg/ml, while in AIF (pH 6.8), its solubility is more than 110-fold less, 0.02 mg/ml.

The recommended ciprofloxacin therapy is dosing two times per day for most indications. The recommended doses and dosing regimens of each drug are described in Physician's Desk Reference 56^{th} Edition (Medical Economics Company, New Jersey, 2002).

A structural polymer carrier comprises a hydrophilic polymer which provides cohesiveness to the blend so durable tablets can be made. The structural polymer also provides during the operation of the delivery system of the present invention a hydrogel with viscosity. This viscosity suspends drug particles to promote partial or complete dissolution of the drug prior to delivery from the dosage form.

If the present invention is used in an erodible matrix application, the molecular weight of the structural polymer is selected to modify the erosion rate of the system. High molecular weight polymers are used to produce slow erosion rate and slow delivery of drug, low molecular weight polymers produce faster erosion rate and faster release of drug. A blend of high and low molecular weight structural polymers produces an intermediate delivery rate.

If the present invention is used in a nonerodible porous matrix, the molecular weight of the structural polymer is selected to provide a hydrogel with viscosity within the pores of the matrix. This viscosity suspends drug particles to promote partial or complete dissolution of the drug in the presence of the solubilizing surfactant prior to delivery from the pores of the dosage form.

A carrier provides a hydrophilic polymer particle in the drug composition that contributes to the controlled delivery of active agent. Representative examples of these polymers are poly(alkylene oxide) of 50,000 to 8 million and more preferably of 100,000 to 750,000 number-average molecular weight, including poly(ethylene oxide), poly(methylene oxide), poly(butylene oxide) and poly(hexylene oxide); and a poly(carboxymethylcellulose) of 40,000 to 1,000,000 400,000 number-average molecular weight, represented by poly(alkali carboxymethylcellulose), poly(sodium carboxymethylcellulose), poly(potassium carboxymethylcellulose) poly(calcium carboxymethylcellulose), and poly(lithium carboxymethylcellulose). The drug composition can comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among those polymers are the poly(ethylene oxide) of 100,000-300,000 number average molecular weight. Carriers that erode in the gastric environment, i.e., bioerodible carriers, are especially preferred.

Other carriers that may be incorporated into drug layer include carbohydrates that exhibit sufficient osmotic activity to be used alone or with other osmoagents. Such carbohydrates comprise monosaccharides, disaccharides and polysaccharides. Representative examples include maltodextrins (i.e., glucose polymers produced by the hydrolysis of grain starch such as rice or corn starch) and the sugars comprising lactose, glucose, raffinose, sucrose, mannitol, sorbitol, zylitol and the like. Preferred maltodextrins are those having a dextrose equivalence (DE) of 20 or less, preferably with a DE ranging from about 4 to about 20, and often 9-20. Maltodextrin having a DE of 9-12 and molecular weight of about 1,600 to 2,500 has been found most useful.

Carbohydrates described above, preferably the maltodextrins, may be used in the drug layer without the addition of an osmoagent, and obtain the desired release of therapeutic agent from the dosage form, while providing a therapeutic effect over a prolonged period of time and up to 24 hours with once-a-day dosing.

Dosage forms in accord with the present invention are manufactured by standard techniques. For example, the dosage form may be manufactured by the wet granulation technique. In the wet granulation technique, the drug, carrier and surfactant are blended using an organic solvent, such as denatured anhydrous ethanol, as the granulation fluid. The remaining ingredients can be dissolved in a portion of the granulation fluid, such as the solvent described above, and this latter prepared solution is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for up to 10 minutes. The composition is pressed into a layer, for example, in a Manesty® press or a Korsch LCT press. For a bilayered core, the drug-containing layer is pressed and a similarly prepared wet blend of the push layer composition, if included, is pressed against the drug-containing layer. The intermediate compression typically takes place under a force of about 50-100 newtons. Final stage compression typically takes place at a force of 3500 newtons or greater, often 3500-5000 newtons. The single or bilayer compressed cores are fed to a dry coater press, e.g., Kilian® Dry Coater press, and subsequently coated with the wall materials as described above. A like procedure is employed for those cores that are manufactured with a push layer and more than one drug layer, typically on a Korsch multi-layer press.

One or more exit orifices are drilled in the drug layer end of the dosage form, and optional water soluble overcoats, which may be colored (e.g., Opadry colored coatings) or clear (e.g., Opadry Clear), may be coated on the dosage form to provide the finished dosage form.

In another manufacture the drug and other ingredients comprising the drug layer are blended and pressed into a solid layer. The layer possesses dimensions that correspond to the internal dimensions of the area the layer is to occupy in the dosage form, and it also possesses dimensions corresponding to the second push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, if included, a layer of osmopolymer composition is placed in contact with the layer of drug in a like manner. The layering of the drug formulation and the osmopolymer layer can be fabricated by conventional two-layer press techniques. The compressed cores then may be coated with the semipermeable wall material as described above.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

An exit is provided in each dosage form. The exit cooperates with the compressed core for the uniform release of drug from the dosage form. The exit can be provided during the manufacture of the dosage form or during drug delivery by the dosage form in a fluid environment of use.

The exit may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

The exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form.

The dosage form can be constructed with one or more exits in spaced-apart relation or one or more surfaces of the dosage form.

Drilling, including mechanical and laser drilling, through the semipermeable wall can be used to form the exit orifice. Such exits and equipment for forming such exits are disclosed in U.S. Pat. No. 3,916,899, by Theeuwes and Higuchi and in U.S. Pat. No. 4,088,864, by Theeuwes, et al. It is presently preferred to utilize a single exit orifice.

### DESCRIPTION OF EXAMPLES OF THE INVENTION

The following examples are illustrative of the present invention and they should not be considered as limiting the scope of the invention in any way, as these examples and other equivalents thereof will become apparent to those versed in the art in light of the present disclosure, drawings and accompanying aspects.

### Example 1

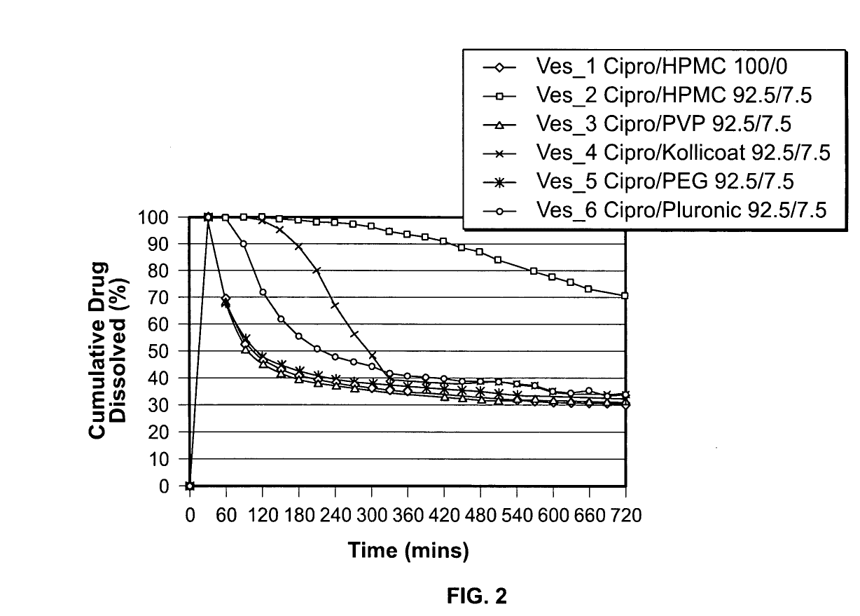

Several dissolution tests were performed to evaluate polymer compositions for their ability to inhibit precipitation of drugs at neutral pH. The precipitation study was conducted using the Distek USP II method. Ciprofloxacin hydrochloride, 500 mg, was first dissolved in 50 mL de-ionized water, pH 5.5. The polymer to be tested was then dissolved in the aqueous drug solution. This clear aqueous drug/polymer solution was added to 850 mL of AIF without enzymes, pH 6.8, to a final weight ratio of 92.5/7.5, ciprofloxacin/polymer. The drug concentration was monitored at 37° C. with a UV spectrophotometer at wavelength of 323 nm. As shown in FIG. 2, hydroxypropyl methyl cellulose (HPMC) was the optimal hydrophilic polymer for preventing drug precipitation. PVP and PEG were indistinguishable from the drug alone. Kollicoat and Pluronic inhibited precipitation less effectively than HPMC.

### Example 2

Ciprofloxacin, HPMC, and Pluronic F108 (Pluronic) were dissolved in de-ionized water at a weight ratio of 90:10:20, respectively. Ciprofloxacin, 10 g, was first added to 400 mL of de-ionized water. When the drug solution turned clear, HPMC and Pluronic were added. The solution was stirred until HPMC and Pluronic were completely dissolved. The clear aqueous ciprofloxacin/HPMC/Pluronic solution was poured onto a flat tray for lyophilization.

The conditions used for lyophilization are summarized in Table 1. After about 2 hours of cooling, the solutions were frozen, a vacuum was applied to the chamber and the drying process started. The total dry time was about 15 hours.

TABLE 1

| | RAMP | HOLD | TIME |
|---|---|---|---|
| SEG | (° C./MN) | (° C.) | (hr) |
| 1 | 1.50 | −34 | 1.0 |
| 2 | 2.53 | +40 | 1.0 |
| 3 | 0.40 | −34 | 1.0 |
| 4 | 1.40 | +0 | 1.0 |
| 5 | 0.70 | +24 | 1.0 |

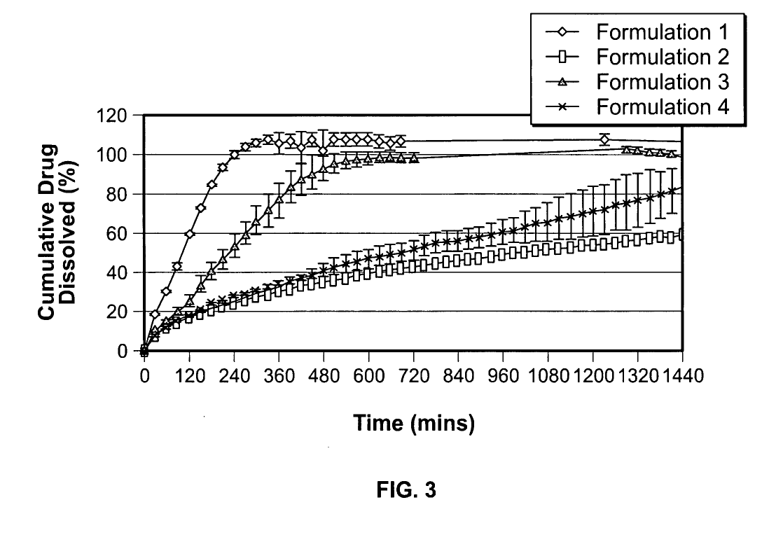

The lyophilized ciprofloxacin/HPMC/Pluronic was passed through a 60 mesh screen. The excipients, including adipic acid, cross-linked carboxymethylcellulose (Acdisol), magnesium stearate, Carbomer 71G and Carbomer 934, were then added to the complex according to the corresponding formulations shown in Table 2. After blending for about 30 minutes, the blend was compresed using oval tooling with one ton of force. The tablets were tested by adding to 900 mL of AIF, pH 6.8, for 24 hours using the USP II paddle method. At different weight ratios of Carbomer 71G to Carbomer 934, the release durations varying from 2 to 24 hrs can be achieved (see FIG. 3). For example, increasing the amount of Carbomer 71G decreased the time to reach the maximal release rate.

TABLE 2
Matrix Tablet Fomulations

| | wt % | | | | Weight (mg) per tablet | | | | |
|---|---|---|---|---|---|---|---|---|---|
| | 1 | 2 | 3 | 4 | 1 | 2 | 3 | 4 | ratio |
| Cipro | 50 | 50 | 50 | 50 | 500 | 500 | 500 | 500 | 90 |
| HPMC E5 | 5.56 | 5.56 | 5.56 | 5.56 | 56.56 | 56.56 | 56.56 | 56.56 | 10 |
| PluronicF108 | 11.11 | 11.11 | 11.11 | 11.11 | 111.11 | 111.11 | 111.11 | 111.11 | 20 |
| Adipic Acid | 15.00 | 15.00 | 15.00 | 15.00 | 150.00 | 150.00 | 150.00 | 150.00 | |
| Acdisol | 10 | 10 | 10 | 10 | 100.00 | 100.00 | 100.00 | 100.00 | |
| Mg Stearate | 0.5 | 0.5 | 0.5 | 0.5 | 5.00 | 5.00 | 5.00 | 5.00 | |
| Carbomer 71G | 7.83 | 0 | 5.22 | 261 | 78.30 | 78.30 | 78.30 | 78.30 | |
| Carbomer 934 | 0 | 7.83 | 261 | 5.22 | 0.00 | 0.00 | 0.00 | 0.00 | |
| Total | 100.00 | 100.00 | 100.00 | 100.00 | 1000 | 1000 | 1000 | 1000 | |

### Example 3

Preparation of Dry-Blended Powder The ingredients in Table 3 for each formulation were combined together in a mixing bowl and mixed dry for about 15 to 30 minutes to produce a dry-blended (DB) ciprofloxacin formulation. The weighted, DB formulations were added to tanks of 900 mL of AIF. The drug concentration was monitored at 37° C. by light absorption at 323 nm using the Distek USP II method.

TABLE 3
Amounts of the Excipients in Each Formulation

| Dry | weight (mg) per tablet | | | | | | |
|---|---|---|---|---|---|---|---|
| Blend | 5 | 6 | 7 | 8 | 9 | 10 | 11 |
| Cipro | 500 | 500 | 500 | 500 | 500 | 500 | 500 |
| HPMC | 55.56 | 55.56 | 55.56 | 0 | 0 | 0 | 55.56 |
| F108 | 0 | 111.11 | 111.11 | 111.11 | 111.11 | 0 | 0 |
| Adipic | 150 | 150 | 0 | 0 | 150 | 0 | 0 |
| acid | | | | | | | |

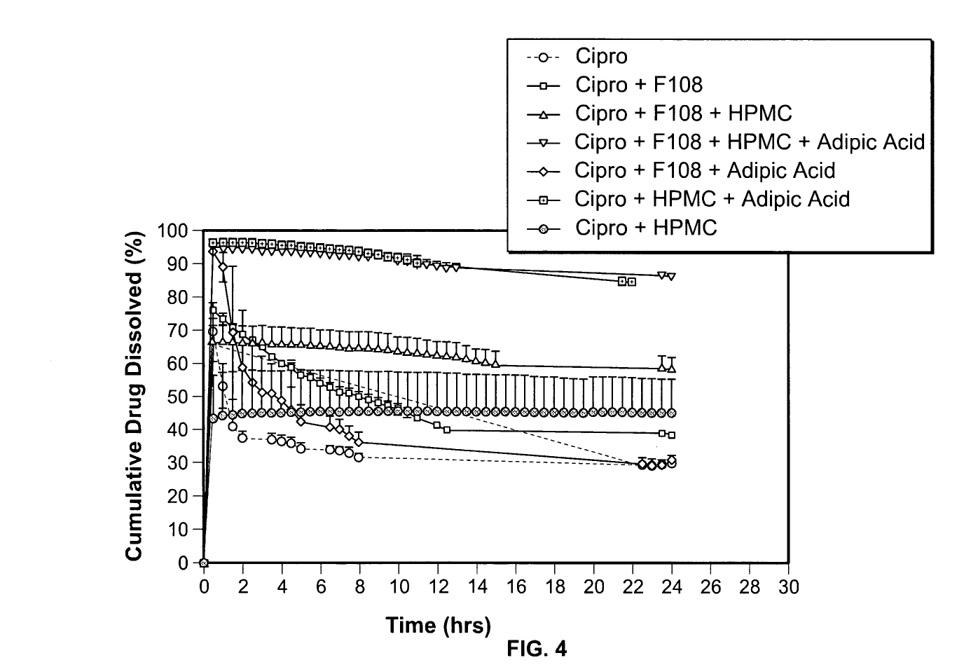

In vitro Testing The results of testing the DB ciprofloxacin formulations (see FIG. 4) show that precipitation of ciprofloxacin in AIF is delayed by more than 20 hours in DB formulations comprising HPMC and adipic acids. In contrast, DB HPMC mixtures without adipic acid perform poorly and DB mixtures without HPMC performed most poorly, with more than 50% of the drug precipitating within the first 10 hours, irrespective of whether adipic acid was present.

### Example 4

Preparation of Freeze-Dried Powder Ciprofloxacin was dissolved in de-ionized (DI) water in the ratio of 40 ml/g of drug. The polymer and excipients were then added to the aqueous drug solution in the amounts shown in Table 4 and stirred until dissolved. The resulting clear solution was poured into flat trays, 400 ml per tray, and transferred to a lyophilizer. Freeze-drying was performed according to the specifications of Table 1. After lyophilization, the resulting dry powder was passed through a 60 mesh screen.

TABLE 4

| | weight (mg) per tablet | | | | | | | | |
|---|---|---|---|---|---|---|---|---|---|
| Freeze-dry | 12 | 13 | 14 | 15 | 16 | 17 | 18 | 19 | 20 |
| Cipro | 500 | 500 | 500 | 500 | 500 | 500 | 500 | 500 | 500 |
| HPMC | 55.56 | 55.56 | 55.56 | 55.56 | 55.56 | 0 | 0 | 0 | 0 |
| F108 | 111.11 | 0 | 111.11 | 0 | 0 | 111.11 | 0 | 0 | 111.11 |
| Adipic acid | 150 | 150 | 0 | 0 | 0 | 150 | 150 | 0 | 0 |
| Solutol | 0 | 0 | 0 | 55.56 | 0 | 0 | 0 | 0 | 0 |

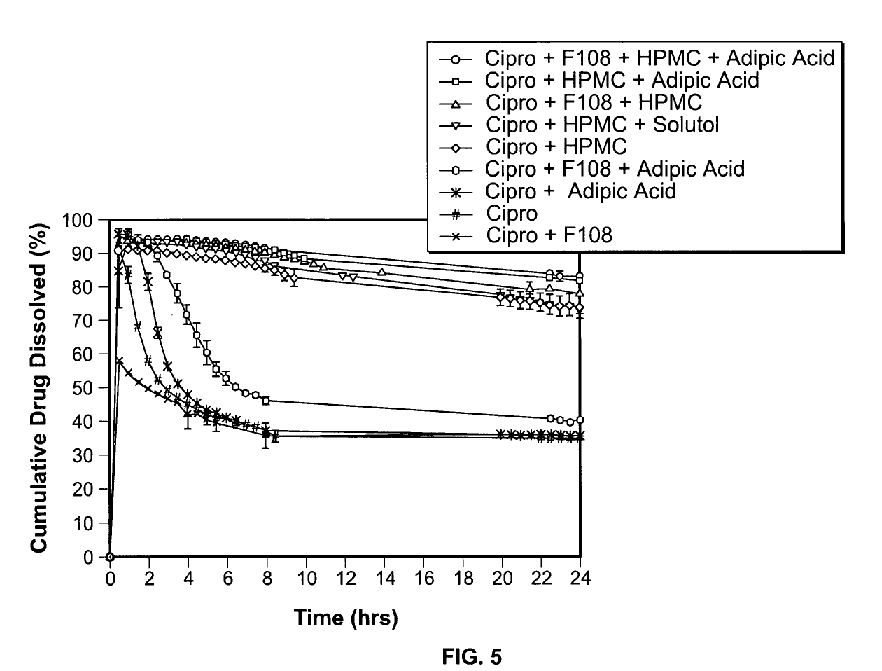

In vitro Testing The freeze-dried (FD) formulations described above (see Table 4, Formulations 12-20) were analyzed in the dissolution assay as described in Example 3. The results obtained with different FD ciprofloxacin formulations (see FIG. 5) show that FD mixtures of ciprofloxacin in with F108/HPMC, F108/HPMC/adipic acid, HPMC/adipic acid, HPMC, and HPMC/Solutol, all delay the precipitation of ciprofloxacin in AIF for more than 20 hours. The results demonstrate that FD formulations comprising HPMC upon dissolution inhibited precipitation of ciprofloxacin in AIF.

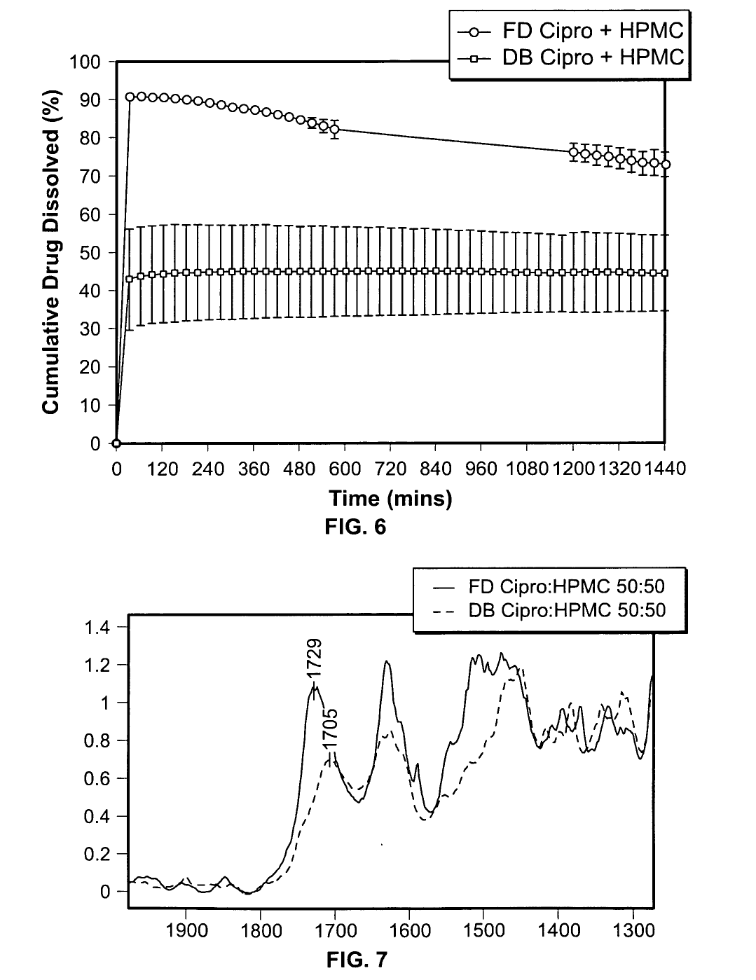

The importance of the complex is heightened when comparing DB and FD formulations comprising ciprofloxacin and HPMC (FIG. 6). The results show that the extent of dissolution is much higher in the FD formulation. This is because the complex increases the solubility of ciprofloxacin and likely increases the rate of dissolution as well.

Characterization of Ciprofloxacin and its HPMC Complex Using FTIR and Raman Spectroscopy FTIR and Raman spectroscopy show that while the FD formulation contains a ciprofloxacin:HPMC complex, the DB formulation does not.

Potassium Bromide (KBr) was mixed with the freeze-dried or dry-blended powder containing ciprofloxacin and HPMC prior to spectroscopy. The mixtures were analyzed by FTIR spectroscopy. The FTIR spectra shows the ν(C═O) stretching mode of the carboxylic acid group of ciprofloxacin occurs as a band at 1705 cm⁻¹. This peak was shifted left to 1729 cm⁻¹ in the FD mixture but was unshifted in the DB mixture (FIG. 7).

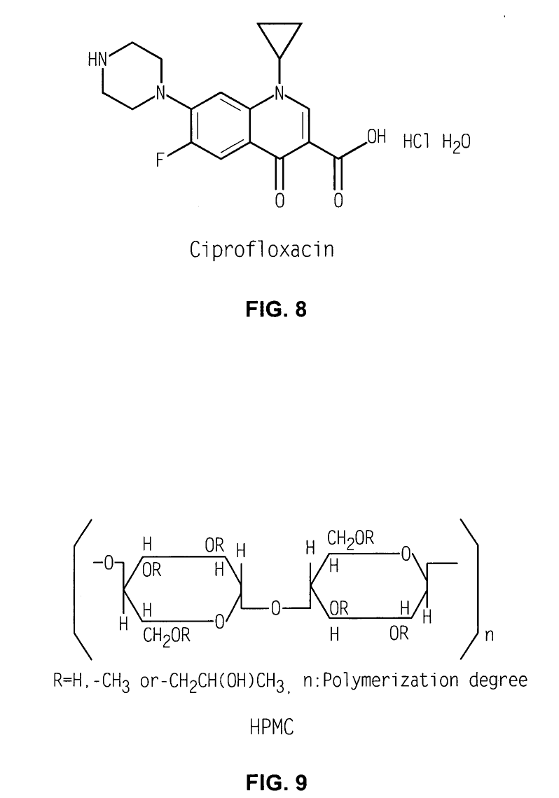

In the complex, the carboxylic acid group of a ciprofloxacin molecule (drawn in FIG. 8) likely hydrogen bonds with the OR group (R:H, CH₃, or CH₂CH(OH)CH₃) of a HPMC molecule (drawn in FIG. 9) and gives a complexed ester carbonyl absorption at about 1729 cm⁻¹.

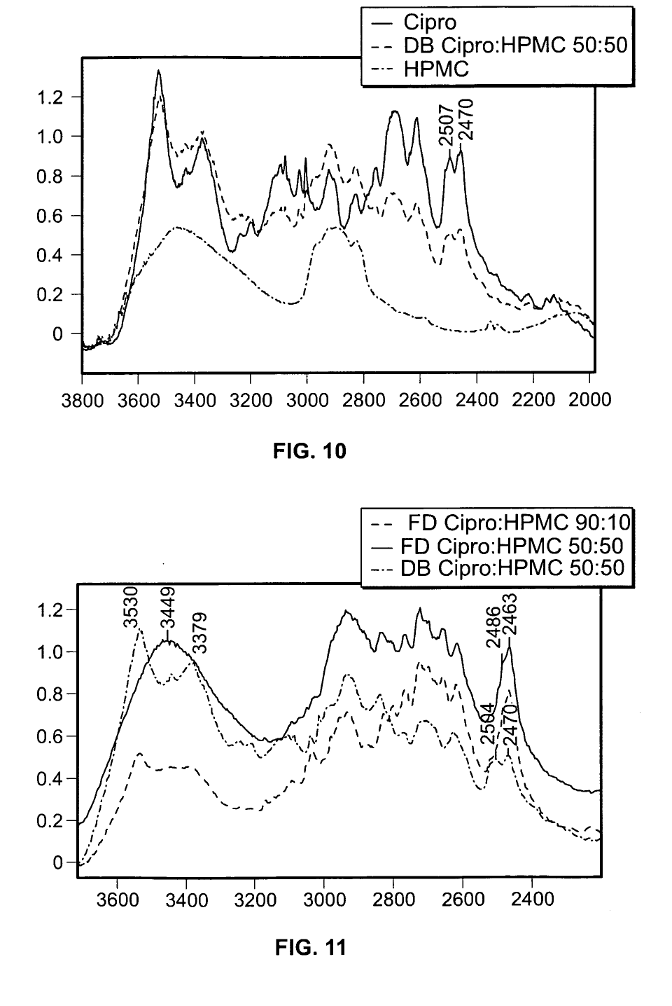

FIGS. 10 and 11 show two FTIR peaks that represent OH stretching of the carboxylic acid group of ciprofloxacin. Ciprofloxacin alone shows two peaks at 2507 cm⁻¹ and 2470 cm⁻¹ (FIG. 10). These bands are unshifted in the DB formulations (FIG. 10, and FIG. 11). In contrast, in the FD ciprofloxacin formulations, one of the peaks is significantly weakened, while both are shifted to the right, to 2486 cm⁻¹ and 2463 cm⁻¹, respectively (FIG. 11). This is again consistent with hydrogen bonding between the carboxylic acid group of ciprofloxacin and an OR group of HPMC weakened the OH bond in the FD formulation and shifted the peak to the right.

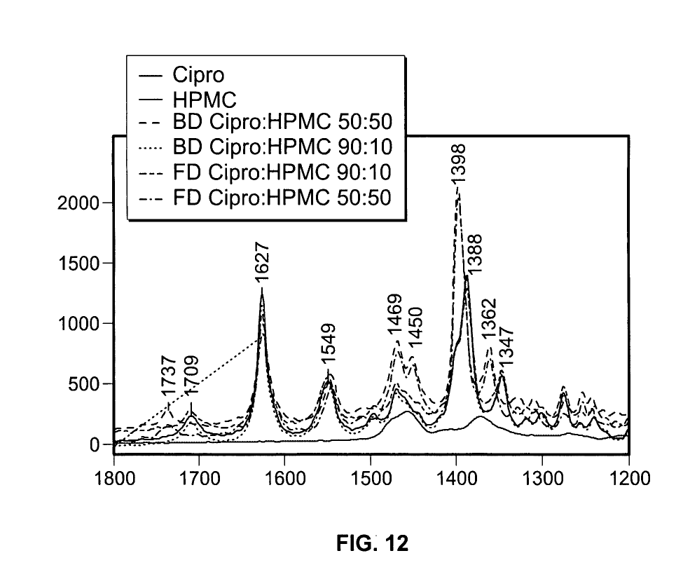

FD and DB powder formulations were prepared and analyzed by Raman spectroscopy using a 534 nm laser. The Raman spectra for ciprofloxacin alone of FIG. 12 show the ν(C═O) stretching mode of the carboxylic acid group of ciprofloxacin occurs as a band at 1709 cm⁻¹; the band at 1627 cm⁻¹ is a pyridone C═O stretch; bands at 1549 cm⁻¹ and 1388 cm⁻¹ are ν(O—C—O) asymmetric and symmetric stretching vibrations, respectively; the band at 1347 cm⁻¹ is a C—N stretching vibration from aromatic amines. These assignments are summarized in Table 5.

TABLE 5
Selected Raman bands comparing ciprofloxacin
alone and in a complex with HPMC (cm⁻¹)

| | v(C═O) | v(C═O) | v(C═O) | v(C═O) | v(C═O) |
|---|---|---|---|---|---|
| | COOH | Pyridone | Asymmetric | Symmetric | Stretching |
| Ciprofloxacin | 1709 | 1627 | 1549 | 1388 | 1347 |
| Dry-Blended | 1709 | 1627 | 1549 | 1388 | 1347 |
| Ciprofloxacin | | | | | |
| Freeze-Dried | 1737 | 1627 | 1546 | 1398 | 1362 |
| Ciprofloxacin: | | | | | |
| HPMC | | | | | |

Similar to FTIR spectra, the Raman spectra of FD formulations of ciprofloxacin:HPMC differ in many respects from spectra obtained with ciprofloxacin alone and from DB mixtures of ciprofloxacin with HPMC (FIG. 12). The ν(C═O) stretching mode of the carboxylic acid group is shifted 28 cm⁻¹. Further, the 1549 cm⁻¹ ν(O—C—O) asymmetric stretching vibration is shifted. The greatest differences between the spectra of FD and DB mixtures are at the 1388 and 1347 cm⁻¹ bands: the1388 cm⁻¹ of ν(O—C—O) symmetric stretching vibration in the Raman spectra is much stronger than the FTIR bands.

In conclusion, Raman and FTIR spectroscopy shows that ciprofloxacin/HPMC complexes do exist and that the complexes are based on hydrogen bonding between the polymer and the drug.

### Example 5

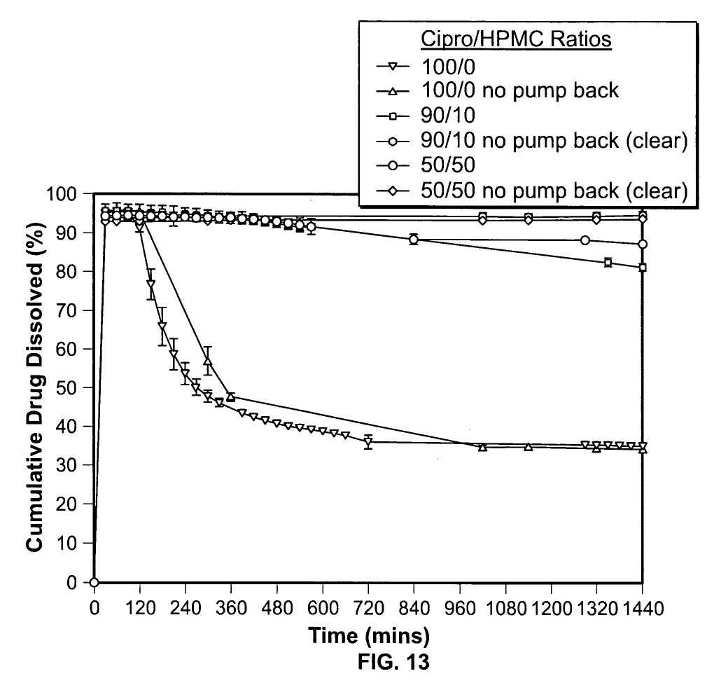

Shear Forces Disrupt the Complex Ciprofloxacin/HPMC were dissolved in 50 ml DI water added to 850 ml pH 6.8 AIF; 100 rpm; 37 C, and ciprofloxacin absorbance at 323 nm was monitored. FIG. 13 shows that a circulation pump moving the aqueous solution created a shear force that caused a decrease in the amount of dissolved drug. Without the pump, both 90/10 and 50/50 ciprofloxacin/HPMC formulations maintain greater than 90% ciprofloxacin dissolved at 24 hours.

These results demonstrate that shear forces can destroy the ciprofloxacin/HPMC complex and causes drug precipitation. In the absence of complexes, the circulation pump does not affect the amount of ciprofloxacin dissolved.

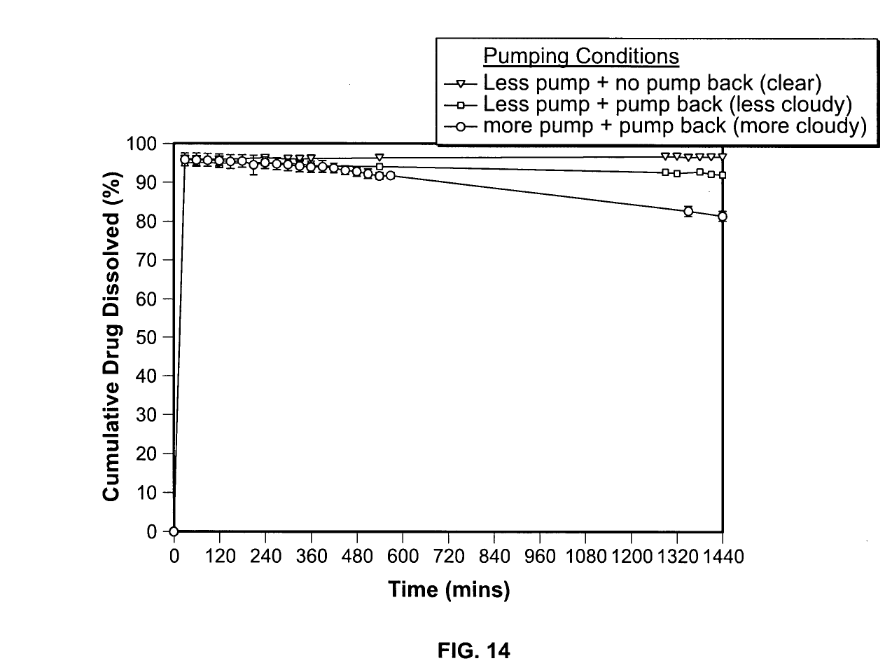

The circulation pump conditions were changed under the same dissolution conditions described above. FIG. 14 shows that gentle circulation through the pump lines gave higher amounts of drug dissolved after 24 hours than vigorous circulation. Absent circulation, the amount of drug dissolved at 24 hours is greater than with circulation. This is likely arising only from inhibiting precipitation by forming a complex.

These results confirm that the complex of ciprofloxacin with the hydrophilic polymer is present in solution, and that the complex inhibits precipitation of ciprofloxacin at neutral pH.

1. A method comprising:
providing a low solubility drug having a pKa between about 6 and about 9;
dissolving the low solubility drug in an aqueous solution, wherein a pH of the aqueous solution is less than about 6.0;
dissolving a hydrophilic polymer in the aqueous solution; and
lyophilizing the aqueous solution to obtain a lyophilized powder, wherein the weight ratio of the hydrophilic polymer to the low solubility drug in the lyophilized powder is less than or equal to about 0.15.

2. The method of claim 1, further comprising:
dissolving the lyophilized powder in a first aqueous solution at pH greater than about 6.0;
measuring precipitation of the low solubility drug from the first aqueous lyophilized powder solution;
wherein the measured precipitation is less than precipitation of the low solubility drug obtained when the low solubility drug is dissolved directly in a second aqueous solution having a pH approximately equal to the first aqueous solution.

3. The method of claim 1, wherein the hydrophilic polymer is selected from the group consisting of hydroxypropyl methylcellulose, methyacrylate ethyl acrylate copolymers and ethylene oxide propylene oxide copolymers.

4. The method of claim 1, wherein the low solubility drug comprises a basic compound.

5. The method of claim 1, wherein the pKa of the low solubility drug is between 6.5 and 7.5.

6. The method of claim 1, wherein the low solubility drug is selected from the group consisting of ciprofloxacin, phenytoin, acyclovir, alprenolol, atenolol, azithromycin, buspirone, carvedilol, diltiazem, imipramine, metoprolol, normorphine, oleandomycin, paromomycin, theophyline and vancomycin.

7. The method of claim 1, wherein the low solubility drug comprises ciprofloxacin.

8. The method of claim 1, wherein FTIR absorbance bands of the low solubility drug present in the lyophilized powder are shifted compared to the low solubility drug alone.

9. The method of claim 1, wherein Raman absorbance bands of the low solubility drug present in the lyophilized powder are shifted compared to the low solubility drug alone.

10. A drug formulation, made according to the method of claim 1.

11. An immediate release dosage form, comprising the drug formulation of claim 10.

12. A controlled release dosage form, comprising the drug formulation oif claim 10.

13. The controlled release dosage form of claim 12, comprising a semipermeable wall, an exit orifice, an expandable layer, and a compacted drug layer, wherein the semipermeable wall is positioned over the at least a portion of the expandable layer, and wherein the compacted drug layer comprises drug formulation of claim 10.

14. The controlled release dosage form of claim 13, wherein the low solubility drug is selected from the group consisting of ciprofloxacin, phenytoin, acyclovir, alprenolol, atenolol, azithromycin, buspirone, carvedilol, diltiazem, imipramine, metoprolol, normorphine, oleandomycin, paromomycin, theophyline and vancomycin.

15. The controlled release dosage form of claim 12, wherein the low solubility drug comprises ciprofloxacin.
